(19) United States
(12) Patent Application Publication
(10) Pub. No.: US 2026/0007788 A1
(43) Pub. Date: Jan. 8, 2026

(54) METHOD AND PRODUCTS FOR GENERATION OF DISINFECTANT COMPOSITIONS

(71) Applicant: Virus Shield Biosciences Limited, Radstock (GB)

(72) Inventor: Craig SHAW, Radstock (GB)

(21) Appl. No.: 18/881,775

(22) PCT Filed: Jul. 7, 2023
(86) PCT No.: PCT/GB2023/051803

§ 371 Date: Jan. 7, 2025

(30) Foreign Application Priority Data
Jul. 7, 2022 (GB) 2209987.3

(51) Int. Cl. A61L 2/00; A61L 2/26; A61L 101/06

(52) U.S. Cl. CPC A61L 2/0088; A61L 2/0094; A61L 2/26; A61L 2101/06

(57) ABSTRACT

A method and products are provided for in situ generation of a disinfectant composition for the treatment of organic matter. The method comprises bringing together a first reagent (12) and a second reagent (14) via a membrane (15) to generate the disinfectant composition. A pouch (10) for use in the method comprises a first section (11) for housing the first reagent (12) and a second section (13) for housing the second reagent (14). The pouch (10) further comprises a membrane (15) adapted in use to permit the first reagent (12) and the second reagent (14) to be brought together, thereby to generate the disinfectant composition. The pouch (10) can be incorporated into the structure of a bag (17) or packaging having a chamber for the organic matter, the pouch (10) being arranged such that the generated disinfectant composition is dispensed into the chamber.

This invention relates, in a first aspect thereof, to a method of in situ generation of a disinfectant composition for the treatment of organic matter. In a second aspect, the invention relates to a pouch for housing reagents used in such a method. And in a third aspect, the invention relates to a bag or packaging material adapted for the generation of a disinfectant composition for the treatment of organic matter contained therein.

The invention has been developed in particular in relation to the generation of chlorine dioxide in liquid, aqueous solution, gas or vapour form for disinfectant purposes, and so will be described herein with particular reference thereto, though it is envisaged that the method and apparatus of the present invention may be adapted for use with other disinfectant liquids, solutions, dispersions, aerosols, gases and vapours.

It is envisaged that both the first and second aspects of the present invention may find use in a wide range of applications where the treatment of organic matter is desirable. Two primary intended uses of the present invention will however be described herein in particular, namely the treatment of agricultural produce prior to packaging and/or consumption, and the treatment of human or animal waste, in particular pet dog faeces, prior to disposal.

The term “treatment” is used broadly herein to encompass disinfection, sterilisation, and the prevention and elimination of mould, mildew, fungi, bacteria, viruses, other pathogens, and odour producing waste products.

Chlorine dioxide is known for its disinfectant properties and has been widely used for bleaching purposes, treatment of drinking water, food processing, disinfection of premises and vehicles, mould eradication, air disinfection, odour control, treatment of swimming pools, dental applications, and wound cleansing. It has been found to be effective as: a disinfectant against Pseudomonas aeruginosa, Staphylococcus aureus, Salmonella enterica, methicillin-resistant S. aureus (MRSA), vancomycin-resistant Enterococcus faecalis, Mycobacterium bovis (TB), Listeria monocytogenes, and Candida albicans; a fungicide against Trichophyton mentagrophytes interdigitale (athlete's foot); a sanitizer against E. coli (and E. coli O157: H7), S. aureus, Salmonella typhimurium (MDRS), Klebsiella pneumonia, and Listeria monocytogenes; against Penicillium digitatum, botrytis Sp, and Fusarium solani; an algaecide against Phormidium boneri; and a virucide against coronavirus, feline calicivirus, hepatitis A virus, human immunodeficiency virus type 1 (HIV-1), poliovirus-1, rotavirus, influenza-A virus, rhinovirus type 37, canine parvovirus, adenovirus type 5, herpes simplex virus type 2, vaccinia virus, norovirus and pandemic 2009 H1N1 influenza A virus (swine flu).

Chlorine dioxide as a disinfectant agent may be deployed in the form of a liquid, gas, vapour or aqueous solution. However, storing and transporting it in these forms can be impractical, costly, inconvenient and hazardous. The present invention seeks to address this issue by providing methods and products for the in-situ generation of disinfectant compositions comprising chlorine dioxide.

According to a first aspect of the present invention there is provided a method of generating a disinfectant composition for the treatment of organic matter, said method comprising bringing together a first reagent and a second reagent via a membrane to generate said disinfectant composition.

In some embodiments of the present invention, the method may be utilised for the treatment of agricultural produce to prevent or eliminate mould, mildew, fungi, bacteria or viruses.

In other embodiments of the present invention, the method may be utilised for the treatment of organic waste, in particular human or animal waste, including pet dog faeces.

The generated disinfectant composition preferably is or comprises chlorine dioxide. This is generated by bringing together a first reagent and a second reagent. The first reagent may preferably be selected from sodium chlorate, sodium chlorite and tetrachlorodecaoxide. Where the first reagent is sodium chlorate, the second reagent is preferably hydrogen peroxide, in the presence of a strong acid. Where the first reagent is sodium chlorite, the second reagent is preferably selected from sodium persulfate, an acid, and an oxidising agent. Where the first reagent is tetrachlorodecaoxide, the second reagent is preferably an acid.

The first and/or second reagents may optionally include one or more additives selected from the group consisting of: disodium peroxodisulphate, citric acid, sodium chloride, cocamidopropyl betaine, ionic surfactants, non-ionic surfactants, cyclopentasiloxane, benzyl ether ethylhexanoate, dimethacone cross polymer, octadecyldimethyl, benzalkonium chloride, siloxanes, dimethyloctadecyl [3-9trimethyoxysilyl) propyl] ammonium chloride, methyl alcohol, and organo siloxanes.

The first reagent and the second reagent may desirably be brought together in the presence of water introduced through the membrane. In this way, the rate of generation, and/or the concentration, of the disinfectant composition can be controlled by the quantity and relative proportions of the first and second reagents, and water, brought together, and/or the porosity of the membrane. The rate of generation, and/or the concentration, of the disinfectant composition may also be selectively controlled according to the type and quantity of organic material to be treated.

The first and second reagents are preferably housed in a pouch comprising a first section housing the first reagent and a second section housing the second reagent. The membrane is preferably provided between the first and second sections, and may effectively define said sections by dividing the pouch. The membrane preferably allows fluid communication between the first and second sections thereby bringing said first and second reagents together.

In this context, it should be understood that the term “bringing together” as utilised herein with reference to the first and second reagents includes enabling fluid communication between said first and second sections in embodiments where said first and second sections are spaced apart, as well as enabling direct physical contact between said first and second sections in embodiments where said first and second sections are directly adjacent one another.

The membrane is preferably provided with at least one removable barrier element adapted to isolate said membrane from the first and/or second sections when not in use. The removable barrier element(s) is adapted to be removed in order to initiate bringing the first and second reagents together.

The pouch may desirably be incorporated into the structure of a bag or packaging for the organic matter. The pouch is preferably arranged such that the generated disinfectant composition is dispensed into a chamber defined within the bag or packaging, in which the organic matter is contained.

In embodiments of the present invention where the organic matter is or comprises organic waste, the method of the present invention may preferably further comprise an additional step of introducing a microbial agent into the chamber, thereby to accelerate the decomposition of the organic waste.

The bag or packaging may preferably be formed from biodegradable material, and the microbial agent may be selected so as to accelerate decomposition of said biodegradable bag or packaging material.

The microbial agent may preferably be housed in a third section adapted to introduce the microbial agent into the chamber. The third section may preferably be provided with a further removable barrier element adapted to isolate said third section from the chamber when not in use. The removable barrier element can then be removed to initiate introducing the microbial agent into the chamber.

According to a second aspect of the present invention, there is provided a pouch for use in a method as hereinbefore described, comprising:
a first section housing a first reagent;
a second section housing a second reagent; and
a membrane adapted in use to permit the first reagent and the second reagent to be brought together, thereby to generate a disinfectant composition for the treatment of organic matter.

In some embodiments, the membrane may divide the pouch so as to effectively to define said first section and said second section.

The pouch may preferably further comprise at least one removable barrier element adapted to isolate the membrane from the first and/or second section when not in use, said removable barrier element(s) being removed when in use to initiate bringing the first and second reagents together.

The pouch is preferably formed from a biodegradable and water permeable cellulose foam material. The membrane is preferably at least partially soluble in water, and may be sewn into the pouch material to divide the first and second section.

The pouch may preferably be supplied pre-loaded with the first and second reagents.

According to a third aspect of the present invention, there is provided a bag or packaging material for organic matter comprising:
a first section housing a first reagent;
a second section housing a second reagent;
a membrane adapted in use to permit the first reagent and the second reagent to be brought together, thereby to generate a disinfectant composition for the treatment of organic matter; and
a chamber, defined within the bag or packaging, for containing said organic matter, the first and second sections being arranged such that the generated disinfectant composition is dispensed into the chamber.

The membrane preferably allows fluid communication between the first and second sections in the presence of water thereby bringing said first and second reagents together. This may be preferably achieved by the membrane being at least partially soluble in water. The water required to dissolve, or partially dissolve, the membrane to initiate bringing together the first and second reagents, and so to initiate the generation of the disinfectant composition, may desirably be provided by water present within the organic matter, without the need for additional water to be added. In preferred embodiments where the bag is intended for the disposal of pet dog faeces, the dissolution or partial dissolution of the membrane to bring together the first and second reagents, and thereby to generate the disinfectant composition, is thus initiated by moisture present within the faeces.

In an alternative embodiment of bag or packaging material according to the third aspect of the present invention, the bag may comprise a pouch as hereinbefore described with reference to the second aspect of the present invention, said pouch housing the first and second sections and the membrane, and being arranged such that the generated disinfectant composition is dispensed into the chamber.

In order that the present invention may be clearly understood, preferred embodiments and applications thereof will now be described in detail, though only by way of example, with reference to the accompanying drawings, in which:

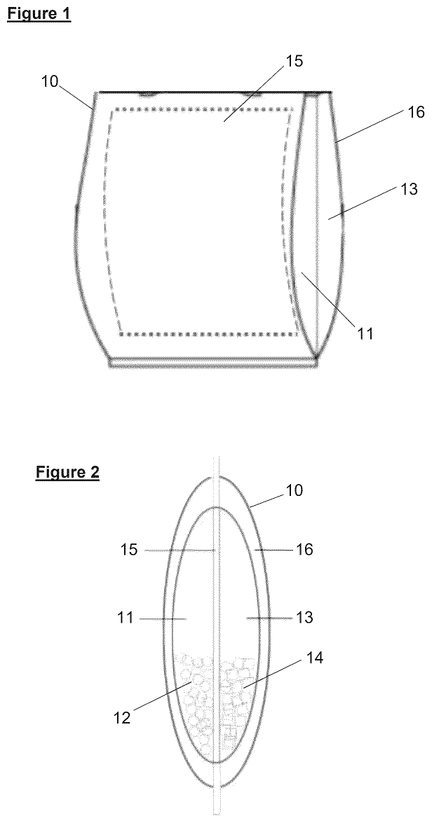

FIG. 1 is a perspective, partially transparent view of a pouch according to the second aspect of the present invention;

FIG. 2 is a side, cross-sectional view of the pouch of FIG. 1;

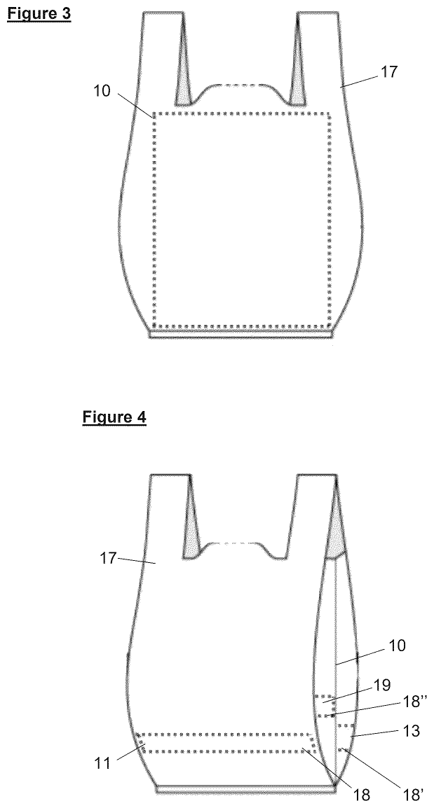

FIG. 3 is a front, partially transparent view of a bag according to the third aspect of the present invention; and

FIG. 4 is a side, partially transparent view of an alternative embodiment of bag according to the third aspect of the present invention.

Referring to FIGS. 1 and 2, in one preferred embodiment of the present invention, a pouch 10 according to the second aspect of the invention is prepared for use in a method according to the first aspect of the invention.

Quantities of first reagent 12 and second reagent 14 appropriate to the application for which the pouch 10 is to be used are calculated based on the desired concentration of disinfectant composition to be generated. The thus determined quantities are accurately weighed and dispensed into, respectively, first 11 and second 13 sections of a pouch 10 formed from a biodegradable and water-permeable cellulose foam material 16.

In between the two sections 11, 13 is provided a perforated membrane 15. The perforations are provided so as in use to control the rate of mixing of the first 12 and second 14 reagents when they are brought together in the presence of water. A ballast weight is also provided within the pouch, the size of the weight depending upon the pouch size. The prepared pouch 10 is sealed inside a moisture free foil envelope for transportation and storage prior to use.

In use, the pouch 10 is removed from the foil envelope and placed in a drum of water. The ballast weight is provided in order to overcome the natural buoyancy of the cellulose pouch 10 and enable it to slowly sink to the bottom of the drum. The water permeates the pouch 10 and the membrane 15, causing dissolution and/or bringing together of the first 12 and second 14 reagents to generate a disinfectant composition comprising chlorine dioxide in aqueous solution. The concentration of the composition can be tailored according to the intended use by varying the volume of water used. Provided the composition is stored in a dark coloured drum, out of direct sunlight, and below 25° C., it can be stored for up to 6 months.

A typical preferred pouch may comprise 71.3 g net weight of active reagents, of which 21.75 g (30.5%) will be sodium chlorite as the first reagent 12, and 49.55 g (69.5%) will be the second reagent 14. Adding this pouch 10 to a 10 litre drum of water will result in an aqueous solution of chlorine dioxide having a concentration of 500 ppm. This can then be used as a stock solution for dilution with further volumes of water where lower concentrations of chlorine dioxide are required, depending on the end application.

Alternative formulations may be selected from the constituents shown in Table 1 below (concentrations as per generated aqueous solution):

TABLE 1

| First Reagent | (g/l) | Second Reagent | (g/l) |
|---|---|---|---|
| sodium chlorate | 0.75 | Hydrogen peroxide + | 0.75 |
| | | strong acid | |
| sodium chlorite | 0.75 | sodium persulfate | 0.75 |
| | | acid | |
| | | oxidising agent | |
| tetrachlorodecaoxide | 0.9 | acid | 0.6 |
| | | oxidising agent | |
| disodium peroxodisulphate | 0.75 | citric acid | 0.75 |
| sodium chloride | 0.75 | citric acid | 0.75 |

The composition may also include one or more optional additives as shown in Table 2 below (percentage weight as per dry composition):

TABLE 2

| Additive | % wt |
|---|---|
| cocamidopropyl betaine | 12% |
| ionic surfactants | 1.5% |
| non-ionic surfactants | 1.5% |
| cyclopentasiloxane | 3% |
| benzyl ether ethylhexanoate | 1% |

The disinfectant composition produced as described above may then be utilised in a range of different applications.

In one intended application, the composition may be dispersed over a target area by fogging or spraying, using a fine high pressure spray system or an electrostatic gun to generate chlorine dioxide gas. At concentrations of chlorine dioxide as low as 5 ppm, this method has been found to kill over 99% of microbial organisms, viruses, and other pathogens. It is also effective against mould & mildew spores. The equipment used should not be set for lower than 50 microns, otherwise aerosolization 10 of the chlorine dioxide solution takes place and the disinfectant effects are diminished.

In another intended application, having several possible uses in agricultural settings, the composition can be applied either directly as a douche or sprayed from handheld trigger sprays. The composition is usually applied and left to evaporate on the surface acting as an oxidiser and cleaning completely the surface. In fruit production, the composition can be used both as a spray and a liquid wash; produce can be mist sprayed during transportation or gassed during storage to remove spores and biofilms, thus protecting the produce from decaying during storage and transportation. As the composition leaves no residue, the produce can proceed straight to market.

In some agricultural applications where the animal or produce is heavily contaminated with soils or faecal matter, the composition can be applied in conjunction with an approved cleaning surfactant-based additive. This surfactant should be plant based and have a neutral pH. The surfactant acts to clear detritus from the surface to be treated and help spread the chlorine dioxide composition across the surface to be treated.

Referring to FIGS. 3, in another preferred embodiment of the present invention, a pouch 10 according to the second aspect of the invention is incorporated into a bag 17 according to the third aspect of the invention. One intended application of the bag 17 is the sterile disposal of animal waste, and in particular pet dog faeces.

In a further variant of this embodiment, as shown in FIG. 4, the first section 11 housing the first reagent 12 and the second section 13 housing the second reagent 14 are provided on opposite sides of the bag 17 and are each provided with a membrane 15 in the form of a water-soluble tape 18. In some variants of this embodiment the tape 18 itself may be impregnated with the reagents 12, 14. As such, the pouch 10 comprises a first tape 18 acting as both the first section 11 and associated membrane 15, and a second tape 18′ acting as both the second section 13 and associated membrane 15. Each tape may preferably be provided with a removable barrier strip.

In use, the first and second tapes 18, 18′ are exposed to moisture in the air, and/or water content in any organic matter such as pet dog faeces contained within the bag 17, by removal of the barrier strips. The tape 18 dissolves upon contact with said water, thus bringing the first and second reagents 12, 14 together, within about 5 minutes. This initiates the generation of chlorine dioxide gas to eliminate pathogenic material within the bag. The gas continues to be produced for around 5 to 10 minutes.

In a further preferred variant of this embodiment, the bag 17 is further provided with a third section 19 housing a microbial agent, provided with a further membrane 15 also in the form of a water-soluble tape 18. Again, the tape 18 itself may be impregnated with the microbial agent, thus acting as both the third section 19 and associated membrane 15, and may preferably be provided with a removable barrier strip. This third tape 18″ preferably has a thicker covering causing it to dissolve slower than the first and second tapes 18, 18′, in around 20 to 30 minutes. By this point the chlorine dioxide gas has acted to eliminate pathogens within the bag 17, and the microbial agent can be released to multiply. The microbial agents acts both to consume the organic waste matter within the bag 17 and to accelerate the decomposition of the biodegradable bag 17.

1. A bag or packaging material for organic matter comprising:
a first section housing a first reagent;
a second section housing a second reagent;
a membrane adapted in use to permit the first reagent and the second reagent to be brought together, thereby to generate a disinfectant composition for the treatment of organic matter; and
a chamber, defined within the bag or packaging, for containing said organic matter, the first and second sections being arranged such that the generated disinfectant composition is dispensed into the chamber.

2. A bag or packaging material as claimed in claim 1, wherein the membrane is at least partially soluble in water, thereby to allow fluid communication between the first and second sections in the presence of water.

3. A bag or packaging material as claimed in claim 1, wherein the first section and the second section are provided on opposite sides of the chamber and are each provided with a membrane in the form of a water-soluble tape.

4. A bag or packaging material as claimed in claim 3, wherein the first section is provided with a first water-soluble tape impregnated with the first reagent, and the second section is provided with a second water-soluble tape impregnated with the second reagent.

5. A bag or packaging material as claimed in claim 3, further comprising a third section housing a microbial agent, provided with a further membrane.

6. A bag or packaging material as claimed in claim 5, wherein the third section is provided with a third water-soluble tape impregnated with the microbial agent.

7. A bag or packaging material as claimed in claim 6, wherein the third water-soluble tape has a thicker covering than the first and second water-soluble tapes, such that said third tape dissolves slower than said first and second tapes.

8. A bag or packaging material as claimed in claim 3, wherein each said tape is provided with a removable barrier strip.

9. A bag or packaging material as claimed in claim 1, for the sterile disposal of human or animal waste, wherein the membrane is adapted at least partially to dissolve in the presence of moisture present within said waste, thereby initiating fluid communication between the first and second sections.

10. A bag as claimed in claim 1, for the sterile disposal of pet dog faeces.

11. A method of in situ generation of a disinfectant composition for the treatment of organic matter, said method comprising bringing together a first reagent and a second reagent via a membrane to generate said disinfectant composition.

12. A method as claimed in claim 11, for the treatment of agricultural produce to prevent or eliminate mould, mildew, fungi, bacteria or viruses.

13. A method as claimed in claim 11, wherein the disinfectant composition is or comprises chlorine dioxide.

14. A method as claimed in claim 13 wherein the first reagent is or comprises sodium chlorite or tetrachlorodecaoxide and the second reagent is or comprises one or more reagents selected from the group consisting of: hydrogen peroxide, sodium persulfate, an acid, and an oxidising agent.

15. A method as claimed in claim 14, wherein the first reagent and the second reagent are brought together in the presence of water introduced through the membrane.

16. A method as claimed in claim 15, wherein the rate of generation, and/or the concentration, of the disinfectant composition is controlled by the quantity and relative proportions of the first and second reagents, and water, brought together, and/or the porosity of the membrane.

17. A method as claimed in claim 16, wherein the rate of generation, and/or the concentration, of the disinfectant composition is selectively controlled according to the type and quantity of organic material to be treated.

18. A method as claimed in claim 11, wherein the first and second reagents are housed in a pouch, said pouch being divided by the membrane into a first section housing the first reagent and a second section housing the second reagent, and wherein the membrane communicates with the first and second sections thereby to bring said first and second reagents together.

19. A method as claimed in claim 18, wherein the membrane is provided with at least one removable barrier element adapted to isolate said membrane from the first and/or second sections, said removable barrier element(s) being removed to initiate bringing the first and second reagents together.

20. A method as claimed in claim 18, wherein the pouch is incorporated into the structure of a bag or packaging for the organic matter.

21. A method as claimed in claim 20, wherein the pouch is arranged such that the generated disinfectant composition is dispensed into a chamber defined within the bag or packaging, in which the organic matter is contained.

22. A method as claimed in claim 21, wherein the organic matter is or comprises organic waste, in particular human or animal waste.

23. A method as claimed in claim 22 further comprising an additional step of introducing a microbial agent into the chamber, thereby to accelerate the decomposition of the organic waste.

24. A method as claimed in claim 23, wherein the bag or packaging is formed from biodegradable material, and wherein the microbial agent further accelerates decomposition of said biodegradable bag or packaging material.

25. A method as claimed in claim 23, wherein the microbial agent is housed in a third section provided with a further removable barrier element adapted to isolate said third section from the chamber defined within the bag or packaging, said removable barrier element being removed to initiate introducing the microbial agent into the chamber.

26. A pouch for use in a method as claimed in claim 11, comprising:
a first section housing a first reagent;
a second section housing a second reagent;
a membrane adapted in use to permit the first reagent and the second reagent to be brought together, thereby to generate a disinfectant composition for the treatment of organic matter.

27. A bag or packaging material for organic matter comprising a pouch as claimed in claim 26, and a chamber, defined within the bag or packaging, for containing said organic matter, the pouch being arranged such that the generated disinfectant composition is dispensed into the chamber.

28. A bag or packaging as claimed in claim 27, further comprising a third section housing a microbial agent and adapted in use to introduce the microbial agent into said chamber.

29. A bag or packaging as claimed in claim 28, formed from biodegradable material, and wherein the microbial agent is adapted on release to accelerate decomposition of said biodegradable bag or packaging material

30. (canceled)
